(19) United States
(12) Patent Application Publication
(10) Pub. No.: US 2026/0350382 A1
(43) Pub. Date: Nov. 5, 2026

(54) BIOMETRY-BASED PERFORMANCE ASSESSMENT

(71) Applicant: CAE Inc., Saint-Laurent (CA)

(72) Inventors: Jean-Francois Delisle, Saint-Laurent (CA); Mark Soodeen, Saint-Laurent (CA)

(73) Assignee: CAE Inc., Saint-Laurent, QC (CA)

(21) Appl. No.: 19/101,892

(22) PCT Filed: Aug. 7, 2023
(86) PCT No.: PCT/CA2023/051054

§ 371 Date: Feb. 6, 2025

Related U.S. Application Data
(60) Provisional application No. 63/370,667, filed on Aug. 7, 2022.

(51) Int. Cl. G06N 3/08

(52) U.S. Cl. CPC G06N 3/08

(57) ABSTRACT

A computerized system for assessing performance based on biometry includes one or more processors executing an artificial intelligence module for correlating, during a model training phase, low-resolution biometric sensor training data obtained from a low-resolution biometric sensor with a cognitive load index generated from high-resolution biometric sensor data to train a cognitive load model. The one or more processors are configured to assess performance during an operational phase by obtaining low-resolution biometric sensor operational data from the low-resolution biometric sensor during the operational phase and determining a cognitive load during the operational phase based on the low-resolution biometric sensor operational data and the cognitive load model.

### CROSS-REFERENCE TO RELATED APPLICATIONS

This application claims priority from U.S. Provisional Patent Application 62/370,667 which is hereby incorporated by reference.

### TECHNICAL FIELD

The present invention relates generally to computer-based systems and computer-implemented methods for training and, more specifically, to computer-based systems and computer-implemented methods for training a student in the operation of a machine such as an aircraft.

### BACKGROUND

Simulation-based training is used to train students in how to operate complex machines such as, for example, how to pilot an aircraft. In most flight simulators, an instructor at an instructor operating station monitors the performance of the student to grade the performance, to provide feedback to the student and to prescribe further lessons. Human monitoring and grading is subjective, prone to oversight, and provides only limited insight into the student's behavior. Biometry has recently been introduced into the realm of flight simulation as a means to provide greater insight into the behavior of the student. Eye tracking is one form of biometry that provides insight into student behavior. Eye tracking may be used to identify areas of interest upon which the student is focused. It would be desirable to provide even greater insight into the behavior of the student in relation to cognitive load. Although some biometric sensors are capable of providing biometry indicative of cognitive load, such as an electroencephalograph, such sensors are considered to be too intrusive to be worn during simulation training. A technical solution to this problem would be highly desirable.

### SUMMARY

In general, the present invention provides a computerized system, method and computer-readable medium for assessing performance by using eye tracking data in order to determine a cognitive load of a student whose performance is being assessed.

One inventive aspect of the disclosure is a computerized system for assessing performance based on biometry. The system includes one or more processors executing an artificial intelligence module for correlating, during a model training phase, low-resolution biometric sensor training data obtained from a low-resolution biometric sensor with a cognitive load index generated from high-resolution biometric sensor data to train a cognitive load model. The one or more processors are configured to assess performance during an operational phase by obtaining low-resolution biometric sensor operational data from the low-resolution biometric sensor during the operational phase and determining a cognitive load during the operational phase based on the low-resolution biometric sensor operational data and the cognitive load model.

In one particular implementation, the system includes an eye tracker for obtaining eye tracking data during a model training phase and a neural activity sensor such as an electroencephalograph (EEG), a functional near-infrared spectroscopy (FNIRS) sensor or a thermal brain imagery sensor, for obtaining neural activity data during the model training phase. One or more processors execute an artificial intelligence module for correlating the eye tracking data with a neural-sensor-derived cognitive load index from a neural-sensor-derived cognitive load model developed from the neural activity data to train, using supervised machine learning, a new, second cognitive load model that can determine cognitive load based on eye tracking data without requiring the neural activity data or other intrusive sensor data. The one or more processors are configured to assess performance during an operational phase by obtaining eye tracking data from the eye tracker during the operational phase and determining a cognitive load during the operational phase based on the eye tracking data and the new, second cognitive load model.

Another inventive aspect of the disclosure is a computer-implemented method of assessing performance based on biometry. The method entails correlating during a model training phase, by an artificial intelligence module, low-resolution biometric sensor training data obtained from a low-resolution biometric sensor with a cognitive load index generated from high-resolution biometric sensor data to train a cognitive load model. The method further entails assessing performance during an operational phase by obtaining low-resolution biometric sensor operational data from the low-resolution biometric sensor during the operational phase and determining a cognitive load during the operational phase based on the low-resolution biometric sensor operational data and the cognitive load model.

In one particular implementation, the method entails training a cognitive load model by obtaining eye tracking data during a model training phase and obtaining neural activity data, e.g. electroencephalogram (EEG) data, functional near-infrared spectroscopy (FNIRS) data or thermal brain imagery data, during the model training phase. The method entails correlating, by an artificial intelligence module, the eye tracking data with a neural-sensor-derived cognitive load index from a first, neural-sensor-derived cognitive load model to train, using supervised machine learning a new, second cognitive load model that can determine cognitive load based on eye tracking data without requiring neural-sensor-data or other intrusive sensor data. The neural-sensor-derived cognitive load index is obtained from an neural-sensor-derived cognitive load model developed from neural-sensor-data. The method further entails assessing performance during an operational phase by obtaining eye tracking data during the operational phase and determining a cognitive load during the operational phase based on the eye tracking data and the new, second cognitive load model.

Another inventive aspect of the disclosure is non-transitory computer-readable medium having instructions in code which are stored on the computer-readable medium and which, when executed by one or more processors of one or more computers, cause the one or more processors to assess performance based on biometry by correlating during a model training phase, by an artificial intelligence module, low-resolution biometric sensor training data obtained from a low-resolution biometric sensor with a cognitive load index generated from high-resolution biometric sensor data to train a cognitive load model. The code causes the one or processors assess performance during an operational phase by obtaining low-resolution biometric sensor operational data from the low-resolution biometric sensor during the operational phase and determining a cognitive load during the operational phase based on the low-resolution biometric sensor operational data and the cognitive load model.

In one particular implementation, a non-transitory computer-readable medium having instructions in code which are stored on the computer-readable medium and which, when executed by one or more processors of one or more computers, cause the one or more computers to assess performance based on biometry by training a cognitive load model by obtaining eye tracking data during a model training phase and obtaining neural activity data, e.g. electroencephalogram (EEG) data, functional near-infrared spectroscopy (FNIRS) data or thermal brain imagery data, during the model training phase. The code also causes the one or more computers to correlate, by an artificial intelligence module, the eye tracking data with an neural-sensor-derived cognitive load index to train, using supervised machine learning, a new, second cognitive load model that can determine cognitive load based on eye tracking data without requiring neural activity data or other intrusive sensor data. The neural-sensor-derived cognitive load index is obtained from an neural-sensor-derived cognitive load model developed from the neural activity data. The code also cases the one or more computers to assess performance during an operational phase by obtaining eye tracking data during the operational phase and determining a cognitive load during the operational phase based on the eye tracking data and the new, second cognitive load model.

The foregoing presents a simplified summary of the invention in order to provide a basic understanding of some aspects of the invention. This summary is not an exhaustive overview of the invention. It is not intended to identify essential, key or critical elements of the invention or to delineate the scope of the invention. Its sole purpose is to present some concepts in a simplified form as a prelude to the more detailed description that is discussed later. Other aspects of the invention are described below in relation to the accompanying drawings.

### BRIEF DESCRIPTION OF THE DRAWINGS

Further features and advantages of the present technology will become apparent from the following detailed description, taken in combination with the appended drawings, in which:

FIG. 1 depicts a system for assessing performance based on biometry in accordance with an embodiment of the present invention;

FIG. 2 depicts a simulation system that may be used in the system of FIG. 1;

FIG. 3A depicts an exemplary data architecture for one particular implementation of the system of FIG. 1;

FIG. 3B depicts data and analytic modules for one particular implementation of the system of FIG. 1;

FIG. 4 is a flowchart of a method of assessing performance based on biometry in accordance with an embodiment of the present invention; and

FIG. 5 depicts an example of a user interface displaying a biometrically based performance assessment of a pilot or student pilot.

It will be noted that throughout the appended drawings, like features are identified by like reference numerals.

### DETAILED DESCRIPTION

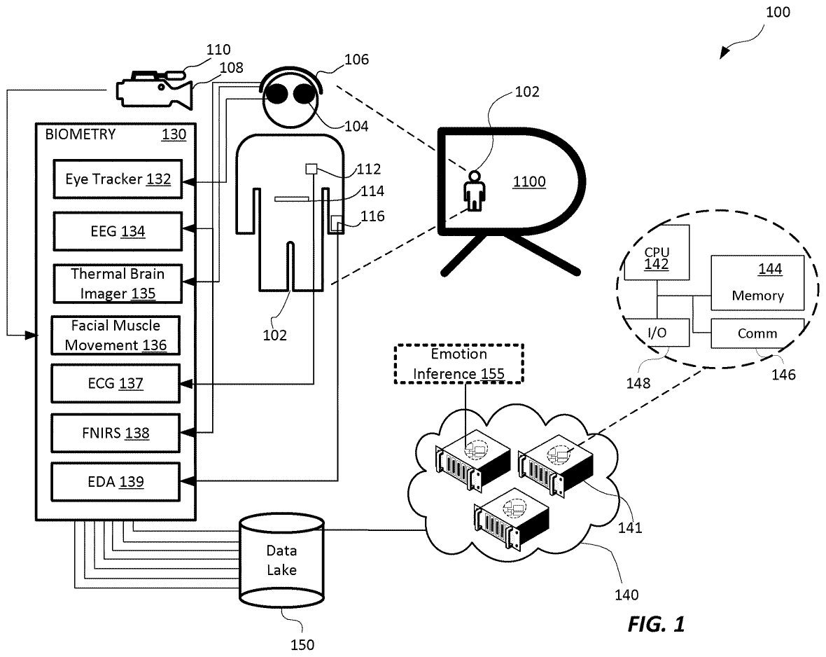

FIG. 1 depicts a computerized system for training a student to operate an actual machine in accordance with an embodiment of the present invention. In this specification, the expression “actual machine” is used to distinguish from a simulated machine that is simulated in a computer simulation to function like the actual machine to thereby train the student in the operation of the actual machine. A flight simulator that simulates the operation of an actual aircraft is one example. The student is a person seeking to learn to operate the actual machine, i.e., a physical and tangible (real-world) machine. The actual machine may be a vehicle such as an aircraft, ship, spacecraft or the like. The actual machine may also be non-vehicular equipment such as a power station, healthcare or medical system, cybersecurity system, or the like. In this specification, the expression “student” is used in an expansive sense to also encompass any person who is training to improve or hone knowledge, skills or aptitude in the operation of the actual machine such as, for example, a licensed pilot who is doing periodic training for certification purposes.

In the embodiment depicted by way of example in FIG. 1, the computerized system is generally designated by reference numeral 100. The computerized system 100 is designed to assess performance of a student pilot based on biometry collected from the student pilot (hereinafter also referred to as simply the student, trainee or operator depending on the particular context). The system 100 may be used as part of a training system or, more particularly an adaptive training system, for training the student 102 to operate an actual machine such as an aircraft. This training may be delivered to the student by providing the student a diverse learning ecosystem (composed of multiple learning environments) that optionally uses an artificial intelligence to adapt to the learning of the student. In the specific example of FIG. 1, the computerized system 100 is a pilot training system for training a student pilot to fly an aircraft. The computerized system 100 may be used, with suitable modifications, to train students to operate other types of vehicular machines such a land vehicles, warships, submarines, spacecraft or to operate non-vehicular machine such as nuclear power stations, cybersecurity command centers, military command centers, etc.

In the embodiment depicted by way of example in FIG. 1, the system 100 is configured to assess the performance of the student 102 by obtaining or collecting biometry (i.e. biometric readings) from the student. In one instance, the system 100 is designed and configured to assess performance based on biometry in order to adapt the training to the learning profile of the student so as to improve the efficiency and efficacy of the training process. The performance assessment may also be used in other ways as will be explained below. Notably, the performance assessment may be used to monitor the performance of licensed pilots while actually flying. This may be used to verify compliance with safety standards, airline performance standards, benchmarks or norms and even to selectively automate certain flight tasks if the performance assessment system detects that the pilot is not performing appropriately or is exhibiting signs of lower cognitive levels.

In the embodiment depicted by way of example in FIG. 1, the system 100 uses artificial intelligence (machine learning techniques) to correlate data from one or more high-resolution biometric sensors (i.e. intrusive, more accurate biometric sensors) with data from one or more low-resolution biometric sensors (non-intrusive, less accurate biometric sensors) for training the cognitive load model and then uses the one or more low-resolution sensors for operations, i.e. for assessing the student or pilot using the trained cognitive load model. As depicted by way of example in FIG. 1, the system 100 includes a low-resolution biometric sensor or a plurality of such low-resolution biometric sensors. The low-resolution biometric sensor may be an eye tracker 132 for obtaining eye tracking data during a model training phase. The eye tracker may be integrated with goggles 104 as shown or within glasses or a visor. The eye tracker may also be mounted within the cockpit without being worn by the student. The low-resolution biometric sensor 104 may alternatively be a facial muscular movement sensor 136 which may include or cooperate with a camera or video camera 108. The low-resolution biometric sensor may alternatively be a low-resolution electroencephalograph (EEG) 134, a low-resolution thermal brain imager 135 or a low-resolution galvanometer of endodermal activity (EDA) sensor or any combination of these sensors. The low-resolution EEG 134 and low-resolution thermal brain imager 135 may be installed within a helmet 106 as shown by way of example. The low-resolution galvanometer of endodermal activity (EDA) sensor may be mounted on a control yoke, cyclic stick or other tangible control instrument of the simulator which the student is holding or touching to thereby measure galvanic skin response or EDA.

In the embodiment of FIG. 1, the high-resolution biometric sensor may be a high-resolution EEG 134, a Functional Near-Infrared Spectroscopy (FNIRS) sensor 138, a high-resolution thermal brain imager 135, a high-resolution galvanometer of endodermal activity (EDA) 139 and an electrocardiogram (ECG) 137 or any combination of these sensors. The FNIRS 138, the ECG 137, the high-resolution EEG 134, the high-resolution thermal brain imager 135 and high-resolution EDA 139 require additional sensors, wiring, setup, etc. and are thus more costly and complicated to set up in the simulator. To clarify, the intrusive high-resolution sensors are more accurate sensors that are not typically worn by a pilot or used in a cockpit in normal flight operations of an aircraft and are considered to be impractical, intrusive, burdensome, distracting and/or uncomfortable, and require additional simulator setup time and cost. In comparison, the non-intrusive low-resolution sensors are less accurate sensors that are frequently or commonly worn by a pilot or used in the cockpit in normal flight operations and which can be worn or used without distracting or burdening the pilot or student. With respect to FIG. 1, for the sake of simplicity, the same numerals are used for the low-resolution and high-resolution versions of the EEG 134, brain imager 135 and EDA 139. For greater clarity, the high-resolution thermal brain imager has a high image resolution (more pixels per unit area) than the low-resolution thermal brain imager. The high-resolution EEG samples more data points per unit time and/or per unit area than a low-resolution EEG. Similarly, the high-resolution galvanometer of endodermal activity (EDA) samples more data points per unit time and/or per unit area than the low-resolution galvanometer of endodermal activity (EDA) sensor.

The model training phase is the phase during which a machine learning model (or artificial intelligence model) is trained. In one implementation, training of the model may be accomplished by using supervised machine learning with low-resolution biometric sensor training data (e.g. eye tracking data such as pupillometry) as a feature and a cognitive load index as a target label. The cognitive load index is determined using high-resolution biometric sensor data. In one specific implementation, a first (base) cognitive load model provides a cognitive load index from high-resolution biometric sensor data (e.g. high-resolution EEG data), which may be obtained elsewhere and/or previously or, alternatively in a secondary implementation, which may be obtained using any one of the high-resolution biometric sensors (e.g. high-resolution EEG). Supervised machine learning is used to train a new, second cognitive load model using low-resolution biometric sensor training data from a low-resolution biometric sensor and the cognitive load index (from the first basic cognitive load model) as a label for the training phase. The low-resolution biometric data is thus correlated with the cognitive load index from a previously established model in order to train, using supervised machine learning, the new, second cognitive load model. The new, second cognitive model is then deployed for use in an operational phase of assessing performance based on low-resolution data e.g. eye tracking data. In other words, during the training phase, non-invasive biometric data such as eye-tracking, facial muscular movement data and/or emotional state as detected by the camera is correlated with cognitive load scores derived from other more intrusive biometric indicators such as high-resolution EEG, FNIRS, high-resolution brain activity thermal imaging data, electrocardiogram (ECG) and/or high-resolution galvanometer of Endodermal activity (EDA) data. In the operational phase, the non-invasive biometric data, such as the eye-tracking data, facial muscular movement data and/or emotional state, is used to determine the cognitive load index or score. In one example implementation using an eye tracker as the low-resolution biometric sensor, during the model training phase, the eye tracker collects eye tracking data that is used for correlation with cognitive load scores or indices obtained from a previously developed base cognitive load model that was previously developed using intrusive high-resolution biometric sensor data (e.g. high-resolution EEG data), FNIRS, ECG or other intrusive high-resolution biometric sensor data. In the model training phase, low-resolution biometric sensor data is collected from the student 102 while the student is performing tasks, e.g. flight maneuvers, in a simulator 1100. For example, if the low-resolution biometric sensor is an eye tracker, eye tracking data is measured. The low-resolution biometric sensor data may be communicatively connected to a biometry data acquisition unit 130. For example, the eye tracker may be communicatively connected to an eye tracker data acquisition unit. Similarly, all of the other low-resolution and high-resolution biometric sensors may be communicatively connected to respective sensor data acquisition units (e.g. an EEG tracker data acquisition unit). The biometry data, or any subset thereof, may be stored in a data lake 150 or other data storage.

As further shown in FIG. 1, one or more processors (e.g. CPU 142) of computing devices 141 may be provided to execute an artificial intelligence module 140 to correlate the low-resolution biometric sensor data with cognitive load indices from the base model to train, using supervised machine learning, a new, second cognitive load model that provides cognitive load scoring based on low-resolution biometric sensor data without requiring a high-resolution sensor like high-resolution EEG or other intrusive sensor data. This new, second cognitive load model is a model that characterizes the cognitive workload of a student based on low-resolution biometric sensor data without requiring intrusive high-resolution biometric sensor data. During simulation training or real-life flight operations, it is undesirable for the student or pilot to wear the high-resolution EEG or other intrusive neural sensor because this is an intrusive sensor whereas the eye tracking sensor (or eye tracker) or other low-resolution biometric sensor is not intrusive. This technology leverages the sensitivity of the high-resolution sensors or other intrusive neural sensors to train the cognitive load model so that the cognitive load model can intelligently interpret the low-resolution biometric sensor data (e.g. eye tracking data) so as to determine or predict cognitive load of the student or pilot based only on the low-resolution biometric sensor data (e.g. eye tracking data). The one or more processors (which may or may be the same one or more processors that trained the model) are configured to assess performance during an operational phase (flight simulation training or actual real-life flying) by obtaining low-resolution biometric sensor data (e.g. eye tracking data from the eye tracker) during the operational phase and predicting a cognitive load during the operational phase based on the low-resolution biometric sensor data (e.g. eye tracking data) and the cognitive load model.

In the system depicted in FIG. 1, the computing devices 141 that form the Al module 141 are depicted as a cluster of servers each having a memory 144 coupled to the CPU 142, communication port 146 and an input/output (I/O) device 148. However, it will be appreciated that a single computing device or any distributed computing system or cloud implementation can be used to implement the Al module.

Furthermore, as depicted in FIG. 1, the system 100 may optionally use groups or subsets of low-resolution and high-resolution biometric sensors to train the model. For example, a plurality of low-resolution biometric sensors that include one or more of the eye tracker, the facial muscular movement sensor, the low-resolution electroencephalograph (EEG), the low-resolution thermal brain imager and the low-resolution galvanometer of endodermal activity (EDA) may be used. Similar, any group or subset of intrusive biometric sensors may be used in the model training phase such as any group or subset of the following intrusive sensors: high-resolution EEG 134, Functional Near-Infrared Spectroscopy (FNIRS) sensor 138, high-resolution thermal brain imager 135, high-resolution galvanometer of endodermal activity (EDA) 139 and electrocardiogram (ECG) 137. Optionally, the system 100 may optionally include in lieu of or in addition to the ECG a heart rate monitor 112 to measure the heart rate (pulse) of the student and/or also an optional blood pressure cuff to monitor the blood pressure of the student. Heart rate and blood pressure are also indicative of stress level or cognitive load. The system may optionally include a respiration monitor 114 to monitor breathing of the student, which is also indicative of stress level and cognitive load. Optionally, the system may include in lieu of or in addition to the EDA sensor a galvanic skin response sensor 116 to measure galvanic skin conductivity due to perspiration, i.e. the emotional or stress-induced response that triggers eccrine sweat gland activity. These sensors 112, 114, 116 are, like the EEG 106, considered to be intrusive sensors that are acceptable to be worn due the training phase of the model but would not be desirable to wear during the operational phase, i.e. during simulation training or actual flying. It will be understood that these optional sensors 112, 114, 116 can be used to refine the cognitive load model but are not essential. The system may also optionally include a thermal imaging camera to measure heat generated by the head of the student in response to elevated stress or workload. The thermal imaging data may be used to supplement as a further parameter in training the model and/or as a further measurement in applying the trained model to assess performance.

In one particular implementation, the low-resolution biometric sensor is an eye tracker that collects eye-related (or vision-related) data of a student or other person whose eyes are being tracked. The eye tracker may track one or both eyes. The eye tracker, in one embodiment, tracks a gaze of the student to provide gaze data. The eye tracker further includes, in one embodiment, a pupillometer to provide pupil dilation data. In one embodiment, the gaze data includes gaze pattern data, fixation data and saccade data or any one of these or any subset of these. The eye tracker may track any one or more of the following: direction of vision (direction of line of sight), focal length (whether the student's eyes are focused on an object that is close or an object that is far), blink rate (where a higher blink rate indicates fatigue and/or higher cognitive workload), and pupil dilation (where dilation of the pupils is indicative of higher cognitive workload). The eye tracker may also provide data relating to a fixation location or an area of interest (AOI) upon which the student is focused or fixated and a fixation duration (how long the student remains focused on the AOI). In the context of flight training, the sequences of fixations on various areas of interest provide useful insight into whether the student pilot is scanning instruments in the desired order and at the desired frequency. The order and frequency of instrument scanning is also maneuver-specific and/or event-specific, e.g. the desired order and frequency of the instrument scanning will depend on the maneuver being performed or the event occurring. The eye tracking data may also provide saccade data. The saccade (rapid eye movements between fixations) are indicative of cognitive workload. A higher number of saccades indicates seeking behavior, i.e. higher workload. The attributes of the eye data are reflective of cognitive workload.

In the system depicted in FIG. 1, the one or more processors may also optionally execute an emotion inference module 155 (which may be part of the Al module 140 or separate module) to infer an emotional state based on a facial expression. Facial expression data may be collected using a video camera 108 and microphone 110. An emotion data acquisition unit may be part of the biometry data acquisition unit 130 as shown by way of example in FIG. 1. Additionally or alternatively, the one or more processors execute an emotion inference module 155 to infer an emotional state based on one or both of voice quality (voice timber indicative of stress of anxiety, i.e. cognitive load) and speech content (vocabulary, grammar and diction, also indicative of cognitive load). In another embodiment, the one or more processors execute an emotion inference module configured to infer an emotional state based on facial muscular movement data. The facial muscular movement data may be captured using a camera. The facial muscular movement data is indicative of emotional states (i.e. taut facial muscles may indicate high stress, fear, anxiety, etc., whereas slack facial muscles may indicate boredom, fatigue, etc.) In one embodiment, the system 100 includes a learner profile. This learner profile may be executed by the Al module 140 in one embodiment. The learner profiles correlates biometrically determined cognition levels with an ability to learn to perform different types of tasks. In the context of flight training, the learner profile may, for example, correlate different types of flight maneuvers or events with different cognition levels to predict how a student will learn a next lesson. The learner profile may cooperate with an adaptive training system to adapt the difficulty or content of the next lesson to the learner profile of the student. For example, if the learner profile reveals that the student exhibits higher than normal stress levels or cognitive workload during a simulated emergency event (e.g. simulated engine seize/fire), then the learner profile can cooperate with the adaptive training system to adapt the training relating to this emergency event to the particular learning profile of this student. This may involve lowering the intensity or other characteristics of the simulated emergency to enable the student to more gradually learn how to cope with the simulated emergency. The learner profile may display to an instructor the cognitive workload of the student to enable the instructor to select alternate or additional training lessons for the student. For example, if the learner profile reveals that the student is overly stressed, the adaptive training system can lower the difficulty of the lesson or of a subsequent lesson. If the learner profiles reveals that the student is disinterested or complacent then the adaptive training system can increase the difficulty of the lesson or of a subsequent lesson. The learner profile may identify events or maneuvers that cause the student undue distress due to the particular psychological makeup of the student which may be useful to adapt training to pinpoint specific areas of weakness. For example, the student may have a latent phobia that trigger panic attacks or causes anxiety levels to spike when certain types of events are simulated. For example, a student with astraphobia (extreme fear of thunder and lightning) may be debilitated during simulation training of a night-time final approach during intense thunder and lightning. The learner profile may be able to discern these spikes of anxiety and to identify that the student is in a debilitating state of extreme anxiety. The instructor can use this learner profile to offer remedial training or help to address the issue.

The biometry data may also be correlated to flight maneuver or events. Examples of flight maneuvers are taxiing, taking off, climbing out, cruising, making a final approach, landing, parking at the gate or jetway, banking, etc. For military jets, examples of flight maneuvers may include airborne re-fueling, carrier takeoff, carrier landings, deploying chaff or flares, launching missiles, bombing, etc. Examples of events are engine seize/fire, fuel leak, electrical fire, smoke in the cabin, loss of cabin pressure, airframe damage, sudden shift in cargo causing an undesirable change in center of gravity, weather event, turbulence, air pocket, strong updraft or downdraft, or sudden fall in headwind or tailwind etc. For each of these maneuvers or events, there are expected ranges of cognitive workload. If a particular student exceeds the expected range for a specific maneuver or event, the learner profile can flag the maneuver or event to the instructor in the IOS for remedial training and/or automatically adapt the training lesson to attempt to improve the student's competency for this particular maneuver or event. Alternatively, instead of determining whether a cognitive load falls within an expected range, the learner profile can flag the maneuver or event if a deviation from a baseline cognitive load for the student exceeds a predetermined threshold. The learner profile can also predict the competency (probability of success or failure) of the student for an upcoming lesson based on maneuver-specific biometry or event-specific biometry. The learner profile can cooperate with the adaptive training system to tailor the learning experience (sequence of lessons, difficulty level, pedagogical content, etc.) based on maneuver-specific biometry or event-specific biometry. Although the foregoing is described in the context of flight training, the biometry collection, profiling and adaptive training may be applied to the training of students in the operation of other types of vehicles (land vehicles, spacecraft, warships, submarines, etc.) or other types of machines (e.g. nuclear power stations, surface-to-air missile defence systems, cybersecurity command centers, etc.).

In the specific context of flight simulation training, the biometry data may be collected from a student in the simulator.

In one embodiment, one or more processors are configured to obtain flight telemetry data for a flight maneuver performed in the simulator. The one or more processors compare the flight telemetry data to a flight maneuver standard prescribed for the flight maneuver to generate flight performance data indicative of a performance of a pilot performing the flight maneuver. The one or more processors correlate the performance of the pilot for the flight maneuver with the eye tracking data to generate a maneuver-specific cognitive load. This correlation may indicate how the pilot is performing during a particular maneuver, e.g. which instruments he is fixated upon, whether there is evidence of unusual saccade and/or blink rate, pupil dilation, stress level, etc., and whether the correct instruments are being scanned for that maneuver in the correct order and with the correct frequency. This enables the one or more processors to assess the pilot performance based on the maneuver-specific cognitive load. For example, the assessment may indicate if the pilot is overloaded during a particular maneuver or if the pilot is too complacent.

In another embodiment, the one or more processors are configured to obtain biometrically based behavior data for an event such as an engine failure or other aircraft malfunction. The one or more processors are configured to compare the biometrically based behavior data for the event to a behaviour standard prescribed for the event to generate behaviour performance data indicative of a behavioral performance of a pilot during the event. The behavior standard presents the behavior expected from the pilot (e.g. situational awareness, communication, crew management). The one or more processors are configured to correlate the behavioral performance of the pilot during the event with the eye tracking data to generate an event-specific cognitive load. The event-specific cognitive load indicates how much mental stress the pilot is under during the event. The one or more processors are configured to assess the pilot based on the event-specific cognitive load. If the stress level is below the expected range, the pilot is too indifferent or complacent. If the stress level is too high above the expected range, the pilot is potentially debilitated by panic or anxiety.

In the embodiments described above, the biometrically based performance assessment is primarily used to provide useful and actionable insight into the student's learning so as to adapt training to the particular profile of the student. However, in another embodiment, the performance assessment may be used in actual, real-life flying to assist the pilot when the pilot is exhibiting a lower level of cognition or when the pilot is cognitively overloaded. In this embodiment, one or more processors execute an augmented cognition module to automate a task otherwise performed by a user in response to detecting a lowered cognitive level of the user. In the context of flying, the augmented cognition module may recognize that the pilot is fatigued, distracted, unwell or mentally overloaded in which case the augmented cognition module may automate one or more flying tasks which were previously being handled by the pilot or co-pilot. This could involve activating an auto-pilot or flight director or activating any other aircraft function such as a deicer, deploying flaps, deploying landing gear, delivering an audio message to passengers or cabin crew, transmitting a report to air-traffic control, etc. The augmented cognition module may be configured to revert to the manual pilot control when it detects that the pilot's biometry is back to normal and the pilot has begun to provide normal input to the flight controls or other aircraft systems. The augmented cognition module may provide audible, visual and tactile alerts to the pilot and co-pilot when it has automated a task and when it has reverted to manual pilot control.

The augmented cognition module may also be used to automate flight operations in response to the biometric sensors (e.g. eye tracker) detecting indicia that are indicative of hypoxia (insufficient level of oxygen in the cockpit). The eye tracker may detect an imminent loss of consciousness of the pilot due to hypoxia by observing ocular attributes such as a blink rate, pupil dilation/contraction, etc.

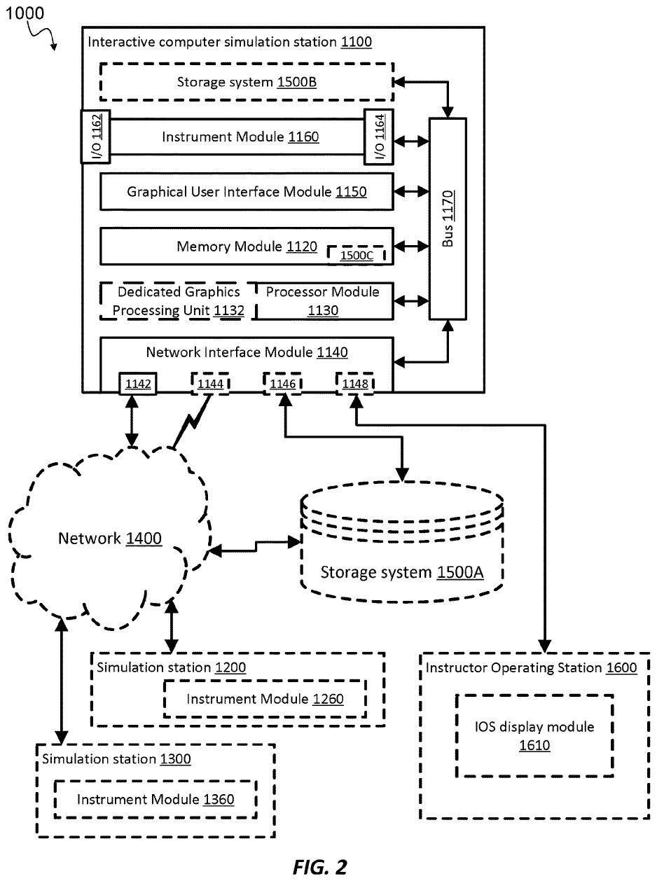

In the embodiment depicted by way of example in FIG. 1, the computerized system 100 includes a simulation station 1100 of a simulation system 1000 shown in FIG. 2 for simulating operation of the actual machine. The simulation system 1000 will now be described in greater detail below in relation to FIG. 2. The simulation station 1100 provides a simulated machine operable in the simulation system by the student. In this particular example, the simulation station 1100 is a flight simulator. As will be described in greater detail below, the system 100 optionally includes a virtual instructor having a coaching Al module and a performance assessment module. The coaching Al module and the performance assessment module respectively coach and assess the student when operating the simulated vehicle in the simulation station 1100. The two modules may be combined into a single module in another embodiment.

As introduced above, the simulation station 1100 shown in FIG. 1 is part of a simulation system 1000 depicted in greater detail in FIG. 2. The simulation system 1000 depicted in FIG. 2 is also referred to herein as an interactive computer simulation system 1000. This simulation system provides an interactive computer simulation of a simulated interactive object (i.e., the simulated machine). The interactive computer simulation system 1000 comprises one or more interactive computer simulation stations 1100, 1200, 1300 which may be executing one or more interactive computer simulations such as a flight simulation software for instance.

In the depicted example of FIG. 2, the interactive computer simulation station 1100 comprises a memory module 1120, a processor module 1130 and a network interface module 1140. The processor module 1130 may represent a single processor with one or more processor cores or an array of processors, each comprising one or more processor cores. In some embodiments, the processor module 1130 may also comprise a dedicated graphics processing unit 1132. The dedicated graphics processing unit 1132 may be required, for instance, when the interactive computer simulation system 1000 performs an immersive simulation (e.g., pilot training-certified flight simulator), which requires extensive image generation capabilities (i.e., quality and throughput) to maintain the level of realism expected of such immersive simulation (e.g., between 5 and 60 images rendered per second or a maximum rendering time ranging between 15 ms and 200 ms for each rendered image). In some embodiments, each of the simulation stations 1200, 1300 comprises a processor module similar to the processor module 1130 and having a dedicated graphics processing unit similar to the dedicated graphics processing unit 1132. The memory module 1120 may comprise various types of memory (different standardized or kinds of Random-Access Memory (RAM) modules, memory cards, Read-Only Memory (ROM) modules, programmable ROM, etc.). The network interface module 1140 represents at least one physical interface that can be used to communicate with other network nodes. The network interface module 1140 may be made visible to the other modules of the computer system 1000 through one or more logical interfaces. The actual stacks of protocols used by physical network interface(s) and/or logical network interface(s) 1142, 1144, 1146, 1148 of the network interface module 1140 do not affect the teachings of the present invention. The variants of the processor module 1130, memory module 1120 and network interface module 1140 that are usable in the context of the present invention will be readily apparent to persons skilled in the art.

A bus 1170 is depicted as an example of means for exchanging data between the different modules of the computer simulation system 1000. The present invention is not affected by the way the different modules exchange information between them. For instance, the memory module 1120 and the processor module 1130 could be connected by a parallel bus, but could also be connected by a serial connection or involve an intermediate module (not shown) without affecting the teachings of the present invention.

Likewise, even though explicit references to the memory module 1120 and/or the processor module 1130 are not made throughout the description of the various embodiments, persons skilled in the art will readily recognize that such modules are used in conjunction with other modules of the computer simulation system 1000 to perform routine as well as innovative steps related to the present invention.

The interactive computer simulation station 1100 also comprises a Graphical User Interface (GUI) module 1150 comprising one or more display screen(s). The display screens of the GUI module 1150 could be split into one or more flat panels, but could also be a single flat or curved screen visible from an expected user position (not shown) in the interactive computer simulation station 1100. For instance, the GUI module 1150 may comprise one or more mounted projectors for projecting images on a curved refracting screen. The curved refracting screen may be located far enough from the user of the interactive computer program to provide a collimated display. Alternatively, the curved refracting screen may provide a non-collimated display.

The computer simulation system 1000 comprises a storage system 1500A-C that may log dynamic data in relation to the dynamic sub-systems while the interactive computer simulation is performed. FIG. 2 shows examples of the storage system 1500A-C as a distinct database system 1500A, a distinct module 1500B of the interactive computer simulation station 1100 or a sub-module 1500C of the memory module 1120 of the interactive computer simulation station 1100. The storage system 1500A-C may also comprise storage modules (not shown) on the interactive computer simulation stations 1200, 1300. The storage system 1500A-C may be distributed over different systems A, B, C and/or the interactive computer simulations stations 1200, 1300 or may be in a single system. The storage system 1500A-C may comprise one or more logical or physical as well as local or remote hard disk drive (HDD) (or an array thereof). The storage system 1500A-C may further comprise a local or remote database made accessible to the interactive computer simulation station 1100 by a standardized or proprietary interface or via the network interface module 1140. The variants of the storage system 1500A-C usable in the context of the present invention will be readily apparent to persons skilled in the art.

An Instructor Operating Station (IOS) 1600 may be provided for allowing various management tasks to be performed in the interactive computer simulation system 1000. The tasks associated with the IOS 1600 allow for control and/or monitoring of one or more ongoing interactive computer simulations. For instance, the IOS 1600 may be used for allowing an instructor to participate in the interactive computer simulation and possibly additional interactive computer simulation(s). In some embodiments, a distinct instance of the IOS 1600 may be provided as part of each one of the interactive computer simulation stations 1100, 1200, 1300. In other embodiments, a distinct instance of the IOS 1600 may be co-located with each one of the interactive computer simulation stations 1100, 1200, 1300 (e.g., within the same room or simulation enclosure) or remote therefrom (e.g., in different rooms or in different locations). Skilled persons will understand that many instances of the IOS 1600 may be concurrently provided in the computer simulation system 1000. The IOS 1600 may provide a computer simulation management interface, which may be displayed on a dedicated IOS display module 1610 or the GUI module 1150. The IOS 1600 may be physically co-located with one or more of the interactive computer simulation stations 1100, 1200, 1300 or it may be situated at a location remote from the one or more interactive computer simulation stations 1100, 1200, 1300.

The IOS display module 1610 may comprise one or more display screens such as a wired or wireless flat screen, a wired or wireless touch-sensitive display, a tablet computer, a portable computer or a smart phone. When multiple interactive computer simulation stations 1100, 1200, 1300 are present in the interactive computer simulation system 1000, the instance of the IOS 1600 may present different views of the computer program management interface (e.g., to manage different aspects therewith) or they may all present the same view thereof. The computer program management interface may be permanently shown on a first of the screens of the IOS display module 1610 while a second of the screen of the IOS display module 1610 shows a view of the interactive computer simulation being presented by one of the interactive computer simulation stations 1100, 1200, 1300). The computer program management interface may also be triggered on the IOS 1600, e.g., by a touch gesture and/or an event in the interactive computer program (e.g., milestone reached, unexpected action from the user, or action outside of expected parameters, success or failure of a certain mission, etc.). The computer program management interface may provide access to settings of the interactive computer simulation and/or of the computer simulation stations 1100, 1200, 1300. A virtualized IOS (not shown) may also be provided to the user on the IOS display module 1610 (e.g., on a main screen, on a secondary screen or a dedicated screen thereof). In some embodiments, a Brief and Debrief System (BDS) may also be provided. In some embodiments, the BDS is a version of the IOS configured to selectively play back data recorded during a simulation session.

The tangible instrument provided by the instrument modules 1160, 1260 and/or 1360 are closely related to the element being simulated. In the example of the simulated aircraft system, for instance, in relation to an exemplary flight simulator embodiment, the instrument module 1160 may comprise a control yoke and/or side stick, rudder pedals, a throttle, a flap switch, a transponder, a landing gear lever, a parking brake switch, and aircraft instruments (air speed indicator, attitude indicator, altimeter, turn coordinator, vertical speed indicator, heading indicator, etc). Depending on the type of simulation (e.g., level of immersivity), the tangible instruments may be more or less realistic compared to those that would be available in an actual aircraft. For instance, the tangible instruments provided by the instrument module(s) 1160, 1260 and/or 1360 may replicate those found in an actual aircraft cockpit or be sufficiently similar to those found in an actual aircraft cockpit for training purposes. As previously described, the user or trainee can control the virtual representation of the simulated interactive object in the interactive computer simulation by operating the tangible instruments provided by the instrument modules 1160, 1260 and/or 1360. In the context of an immersive simulation being performed in the computer simulation system 1000, the instrument module(s) 1160, 1260 and/or 1360 would typically replicate of an instrument panel found in the actual interactive object being simulated. In such an immersive simulation, the dedicated graphics processing unit 1132 would also typically be required. While the present invention is applicable to immersive simulations (e.g., flight simulators certified for commercial pilot training and/or military pilot training), skilled persons will readily recognize and be able to apply its teachings to other types of interactive computer simulations.

In some embodiments, an optional external input/output (I/O) module 1162 and/or an optional internal input/output (I/O) module 1164 may be provided with the instrument module 1160. Skilled people will understand that any of the instrument modules 1160, 1260 and/or 1360 may be provided with one or both of the I/O modules 1162, 1164 such as the ones depicted for the computer simulation station 1100. The external input/output (I/O) module 1162 of the instrument module(s) 1160, 1260 and/or 1360 may connect one or more external tangible instruments (not shown) therethrough. The external I/O module 1162 may be required, for instance, for interfacing the computer simulation station 1100 with one or more tangible instruments identical to an Original Equipment Manufacturer (OEM) part that cannot be integrated into the computer simulation station 1100 and/or the computer simulation station(s) 1200, 1300 (e.g., a tangible instrument exactly as the one that would be found in the interactive object being simulated). The internal input/output (I/O) module 1162 of the instrument module(s) 1160, 1260 and/or 1360 may connect one or more tangible instruments integrated with the instrument module(s) 1160, 1260 and/or 1360. The I/O module 1162 may comprise necessary interface(s) to exchange data, set data or get data from such integrated tangible instruments. The internal I/O module 1162 may be required, for instance, for interfacing the computer simulation station 1100 with one or more integrated tangible instruments that are identical to an Original Equipment Manufacturer (OEM) part that would be found in the interactive object being simulated. The I/O module 1162 may comprise necessary interface(s) to exchange data, set data or get data from such integrated tangible instruments.

The instrument module 1160 may comprise one or more tangible instrumentation components or subassemblies that may be assembled or joined together to provide a particular configuration of instrumentation within the computer simulation station 1100. As can be readily understood, the tangible instruments of the instrument module 1160 are configured to capture input commands in response to being physically operated by the user of the computer simulation station 1100.

The instrument module 1160 may also comprise a mechanical instrument actuator 1166 providing one or more mechanical assemblies for physical moving one or more of the tangible instruments of the instrument module 1160 (e.g., electric motors, mechanical dampeners, gears, levers, etc.). The mechanical instrument actuator 1166 may receive one or more sets of instruments (e.g., from the processor module 1130) for causing one or more of the instruments to move in accordance with a defined input function. The mechanical instrument actuator 1166 of the instrument module 1160 may alternatively, or additionally, be used for providing feedback to the user of the interactive computer simulation through tangible and/or simulated instrument(s) (e.g., touch screens, or replicated elements of an aircraft cockpit or of an operating room). Additional feedback devices may be provided with the computing device 1110 or in the computer system 1000 (e.g., vibration of an instrument, physical movement of a seat of the user and/or physical movement of the whole system, etc.).

The interactive computer simulation station 1100 may also comprise one or more seats (not shown) or other ergonomically designed tools (not shown) to assist the user of the interactive computer simulation in getting into proper position to gain access to some or all of the instrument module 1160.

In the depicted example of FIG. 2, the interactive computer simulation station 1100 shows optional interactive computer simulation stations 1200, 1300, which may communicate through the network 1400 with the simulation computing device. The stations 1200, 1300 may be associated to the same instance of the interactive computer simulation with a shared computer-generated environment where users of the computer simulation stations 1100, 1200, 1300 may interact with one another in a single simulation. The single simulation may also involve other simulation computer simulation stations (not shown) co-located with the computer simulation stations 1100, 1200, 1300 or remote therefrom. The computer simulation stations 1200, 1300 may also be associated with different instances of the interactive computer simulation, which may further involve other computer simulation stations (not shown) co-located with the computer simulation station 1100 or remote therefrom.

In the context of the depicted embodiments, runtime execution, real-time execution or real-time priority processing execution corresponds to operations executed during the interactive computer simulation that may have an impact on the perceived quality of the interactive computer simulation from a user perspective. An operation performed at runtime, in real time or using real-time priority processing thus typically needs to meet certain performance constraints that may be expressed, for instance, in terms of maximum time, maximum number of frames, and/or maximum number of processing cycles. For instance, in an interactive simulation having a frame rate of 60 frames per second, it is expected that a modification performed within 5 to 10 frames will appear seamless to the user. Skilled persons will readily recognize that real-time processing may not actually be achievable in absolutely all circumstances in which rendering images is required. The real-time priority processing required for the purpose of the disclosed embodiments relates to the perceived quality of service by the user of the interactive computer simulation and does not require absolute real-time processing of all dynamic events, even if the user was to perceive a certain level of deterioration in the quality of the service that would still be considered plausible.

A simulation network (e.g., overlaid on the network 1400) may be used, at runtime (e.g., using real-time priority processing or processing priority that the user perceives as real-time), to exchange information (e.g., event-related simulation information). For instance, movements of a vehicle associated with the computer simulation station 1100 and events related to interactions of a user of the computer simulation station 1100 with the interactive computer-generated environment may be shared through the simulation network. Likewise, simulation-wide events (e.g., related to persistent modifications to the interactive computer-generated environment, lighting conditions, modified simulated weather, etc.) may be shared through the simulation network from a centralized computer system (not shown). In addition, the storage module 1500A-C (e.g., a networked database system) accessible to all components of the computer simulation system 1000 involved in the interactive computer simulation may be used to store data necessary for rendering the interactive computer-generated environment. In some embodiments, the storage module 1500A-C is only updated from the centralized computer system and the computer simulation stations 1200, 1300 only load data therefrom.

The computer simulation system 1000 of FIG. 2 may be used to simulate the operation by a user of a user vehicle. For example, in a flight simulator, the interactive computer simulation system 1000 may be used to simulate the flying of an aircraft by a user acting as the pilot of the simulated aircraft. In a battlefield simulator, the simulator may simulate a user controlling one or more user vehicles such as airplanes, helicopters, warships, tanks, armored personnel carriers, etc. In both examples, the simulator may simulate an external vehicle (referred to herein as a simulated external vehicle) that is distinct from the user vehicle and not controlled by the user.

The simulation system 1000 is only one example implementation. It will be appreciated that other types of simulation systems 1000 may be used with the biometry-based performance assessment system 100.

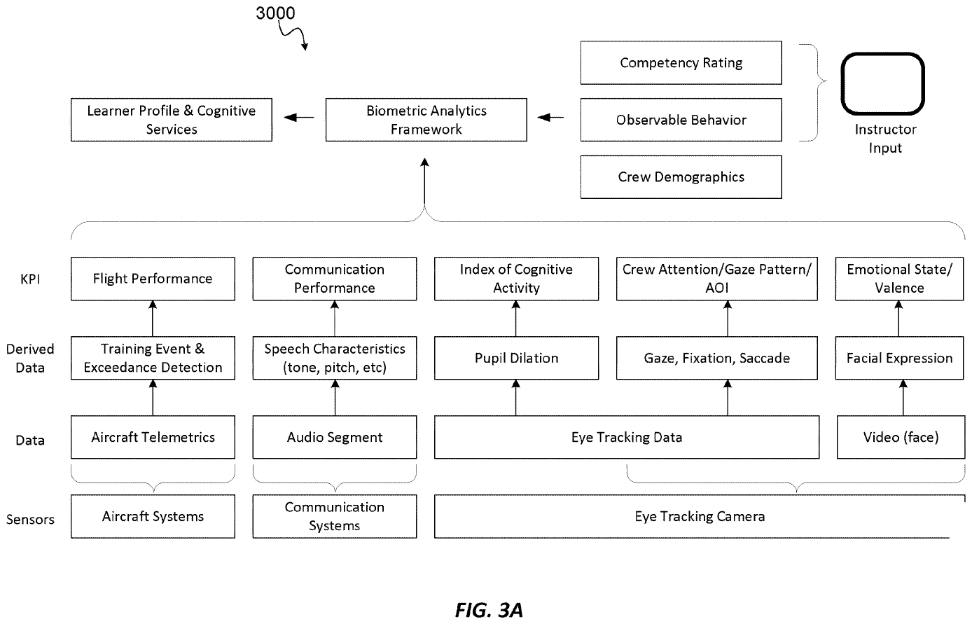

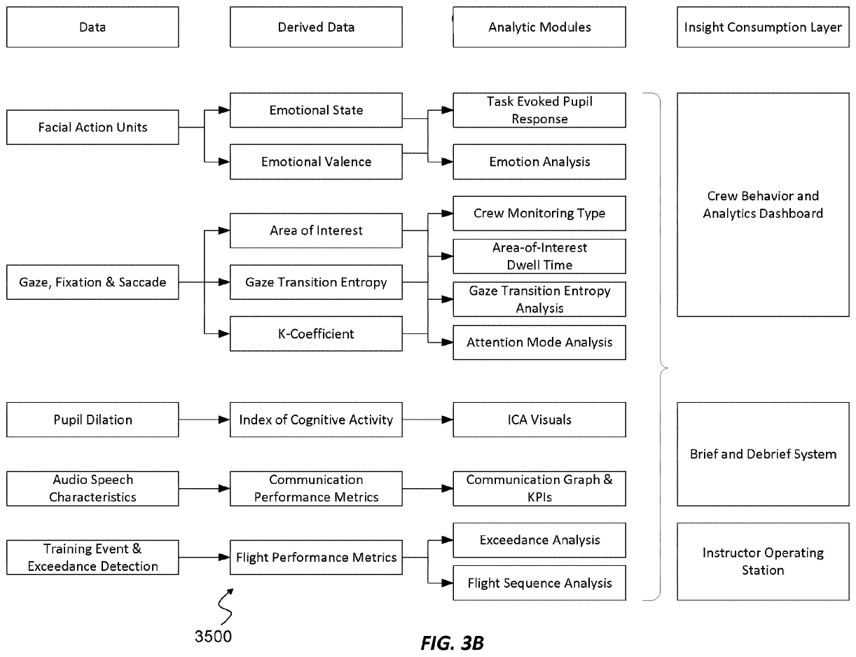

The biometry-based performance assessment system 100 may have various modules to perform various data collection, data analysis and data interpretation functions. FIG. 3A depicts one exemplary data architecture 3000 for one specific implementation of the biometry-based performance assessment system 100. As depicted in FIG. 3A, the biometry-based performance assessment system 100 has a biometric analytics framework that provides results to a learner profile and cognitive services module. The biometric analytics framework receives input from a competency rating module and an observable behavior module (which, in turn, receive input from an instructor). A crew demographics module may also supply data to the biometric analytics framework. The biometric analytics framework receives various forms of data as shown in FIG. 3A. Sensors collect data from aircraft systems, communication systems and one or more eye tracking camera(s). Aircraft system sensors provide aircraft telemetrics (or aircraft telemetry). Communication system sensors provide audio segment data. Eye tracking cameras provide eye-tracking data and video for facial analysis. This raw data layer can be supplemented with additional data. Above the raw data layer is a derived data layer as shown in FIG. 3A. Training event and exceedance data may be derived from the aircraft telemetry. Speech characteristics (e.g. tone, pitch, timber, etc.) may be derived from the audio segment data. Pupil dilation may be derived from the eye tracking data. Gaze, fixation and saccade data may also be derived from the eye tracking data. Facial expression data may be derived from the video camera data. As further shown in FIG. 3A, key performance indicators (KPI) may be determined from the derived data. Flight performance may be determined from the training event and exceedance data. Communication performance may be determined from the speech characteristics. Index of cognitive activity may be determined from the pupil dilation. Crew attention, gaze pattern and AOI may be determined from the gaze, fixation and saccade data. Emotional state and valence may be determined from the facial expression.

FIG. 3B depicts data and analytic modules for another implementation of the biometry-based performance assessment system 100 of FIG. 1. As depicted in FIG. 3B, there are various types of data and derived data as well as analytic modules and an insight consumption layer. In this implementation, the data includes (i) facial action units, (ii) gaze, fixation and saccade data; (iii) pupil dilation data; (iv) audio speech characteristics; (v) training event and exceedance detection. The derived data is similar to what was presented in FIG. 3A with some additional types of derived data. Flight performance metrics are derived from training event and exceedance detection. Communication performance metrics are derived from audio speech characteristics. An index of cognitive activity is derived from pupil dilation. An area of interest is derived from gaze, fixation and saccade data. Gaze transition entropy is also derived from gaze, fixation and saccade data. Furthermore, a K-coefficient (indicative of whether the gaze is focal versus ambient) is also derived from gaze, fixation and saccade data. Emotional state is derived from facial action units. Emotional valence is also derived from facial action units. As further depicted in FIG. 3B, flight performance metrics can be analyzed by an exceedance analysis module and a flight sequence analysis module. Communication performance metric can be analyzed by a communication graph and KPI module. The index of cognitive activity (ICA) can be analyzed and presented to a student or instructor by a ICA Visualization module. The area of interest (AOI), gaze transition entropy (GTE) and K-coefficient can be used by various analytic modules to provide information and insight about cognitive load such as, for example, a crew monitoring type, an AOI dwell time, GTE analysis, attention mode analysis. Emotional state and emotional valence can be analyzed to provide a task-evoked pupil response and an emotion analysis. At the insight consumption layer, as shown in FIG. 3B, there is a crew behavior and analytics dashboard, a brief and debrief system and an instructor operating station (IOS) which can be used by the instructor and student to review the performance assessment and to manage and adapt further training.

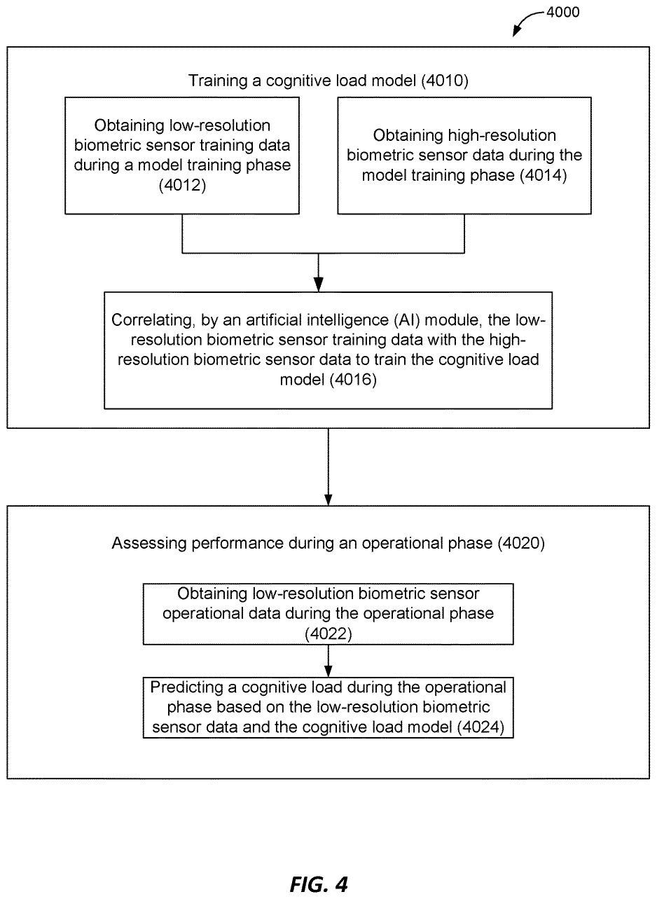

Another aspect of the invention is a computer-implemented method of assessing performance based on biometry. In general, the method uses a previously developed base model (referred to herein as an EEG-derived cognitive load model) that converts EEG data into cognitive load indices or scores. This EEG-derived cognitive load model is applied to captured EEG data to derive labels for training data. The training data includes low-resolution biometric sensor data, e.g. eye tracking data and optionally other forms of low-res sensor data deemed reliable indicators of cognitive load, such as data provided by thermal imaging cameras (FLIR), to generate a machine learning model. In operation, the trained machine learning model, which can, for instance, be a support vector machine (SVM), a convolutional neural network (CNN), or an extreme gradient boosting (XG Boost) algorithm, takes the low-resolution biometric sensor data (eye tracking data and any other non-invasive, low-resolution biometric data on which the model was trained) to output a cognitive load index. The method is outlined in a flowchart in FIG. 4. As depicted in the flowchart of FIG. 4, the method 4000 entails a step, act or operation 4010 of training a cognitive load model. The training is accomplished by obtaining low-resolution biometric sensor training data during a model training phase (step 4012) and obtaining a cognitive load index of a base model that has been developed from correlating cognitive loads with high-resolution biometric sensor data (step 4014). These steps 4012, 4014 should be performed substantially contemporaneously so that the biometric data reflecting the state of the student is obtained at the same point in time so that the cognitive load index can be correlated with the low-resolution biometric sensor data. The method 4000 further entails a step 4016 of correlating, by an artificial intelligence (Al) module, the low-resolution biometric sensor data with the cognitive load index to train the new cognitive load model using supervised machine learning. The method 4000 further entails a step 4020 of assessing performance during an operational phase. The operational phase may be the phase where a student or trainee is learning to perform a task on a simulator. Alternatively, the operational phase may be the phase where a pilot or other machine operator is flying a real aircraft or operating an actual machine. In both cases, the performance assessment is conducted to determine how well the student, trainee, pilot or operator is performing. To clarify, this performance assessment technology can be used both for training a student on a simulator and for evaluating an operator in a real-life scenario. The step 4020 is accomplished by obtaining low-resolution biometric sensor operational data during the operational phase at step 4022. The low-resolution biometric sensor operational data is then used to assess performance using the model that was previously trained. In other words, the step 4020 is accomplished by determining a cognitive load during the operational phase based on the low-resolution biometric sensor operational and the cognitive load model (at step 4024).

Optionally, the method entails obtaining eye tracking data as a form of low-resolution biometric sensor data by obtaining gaze data from an eye tracker and obtaining pupil dilation data from a pupillometer. The gaze data may include gaze pattern data, fixation data and saccade data. In a variant, the gaze data may include any one or subset of the pattern data, fixation data and saccade data. Other eye tracking data may be utilized as well.

Optionally, the method may be enhanced using an emotion inference module to infer an emotional state based on a facial expression and using Facial Action Units in a Facial Action Coding System (FACS). Additionally or alternatively, the emotion inference module may be configured to infer the emotional state based on one or both of voice quality and speech content. Voice quality for this specification is to be understood as any voice characteristic that is indicative of stress or anxiety such as timber, volume, rate, pitch and tone. Speech content for this specification is meant to be understood as the linguistic expression (vocabulary, choice of words, grammar, diction, etc.) that reveal the anxiety level of the speaker.

In one embodiment, the method includes generating a learner profile that correlates biometrically determined cognition levels with ability to learn to perform different types of tasks.

In one embodiment, the method includes an augmented cognition module to automate a task otherwise performed by a user in response to detecting a lowered cognitive level of the user.

These methods can be implemented in hardware, software, firmware or as any suitable combination thereof. That is, if implemented as software, the computer-readable medium comprises instructions in code which when loaded into memory and executed on a processor of a computing device causes the computing device to perform any of the foregoing method steps. These method steps may be implemented as software, i.e. as coded instructions stored on a computer readable medium which performs the foregoing steps when the computer readable medium is loaded into memory and executed by the microprocessor of the computing device. A computer readable medium can be any means that contain, store, communicate, propagate or transport the program for use by or in connection with the instruction execution system, apparatus or device. The computer-readable medium may be electronic, magnetic, optical, electromagnetic, infrared or any semiconductor system or device. For example, computer executable code to perform the methods disclosed herein may be tangibly recorded on a computer-readable medium including, but not limited to, a floppy-disk, a CD-ROM, a DVD, RAM, ROM, EPROM, Flash Memory or any suitable memory card, etc. The method may also be implemented in hardware. A hardware implementation might employ discrete logic circuits having logic gates for implementing logic functions on data signals, an application-specific integrated circuit (ASIC) having appropriate combinational logic gates, a programmable gate array (PGA), a field programmable gate array (FPGA), etc. For the purposes of this specification, the expression “module” is used expansively to mean any software, hardware, firmware, or combination thereof that performs a particular task, operation, function or a plurality of related tasks, operations or functions. When used in the context of software, the module may be a complete (standalone) piece of software, a software component, or a part of software having one or more routines or a subset of code that performs a discrete task, operation or function or a plurality or related tasks, operations or functions. Software modules have program code (machine-readable code) that may be stored in one or more memories on one or more discrete computing devices. The software modules may be executed by the same processor or by discrete processors of the same or different computing devices.

The methods and systems described above enable a highly accurate biometry-based performance assessment to be achieved without the use of intrusive biometric sensors such as an EEG which would be undesirable for a student or pilot to wear during an operational phase (during simulation training in a simulator or during actual real-life flight operations in an actual aircraft). These methods and systems can be used by the instructor to provide insightful feedback to the student about his performance. These methods and systems also enable an adaptive training system to adapt a current lesson and/or to adapt future lessons based on the biometry-based performance assessment.

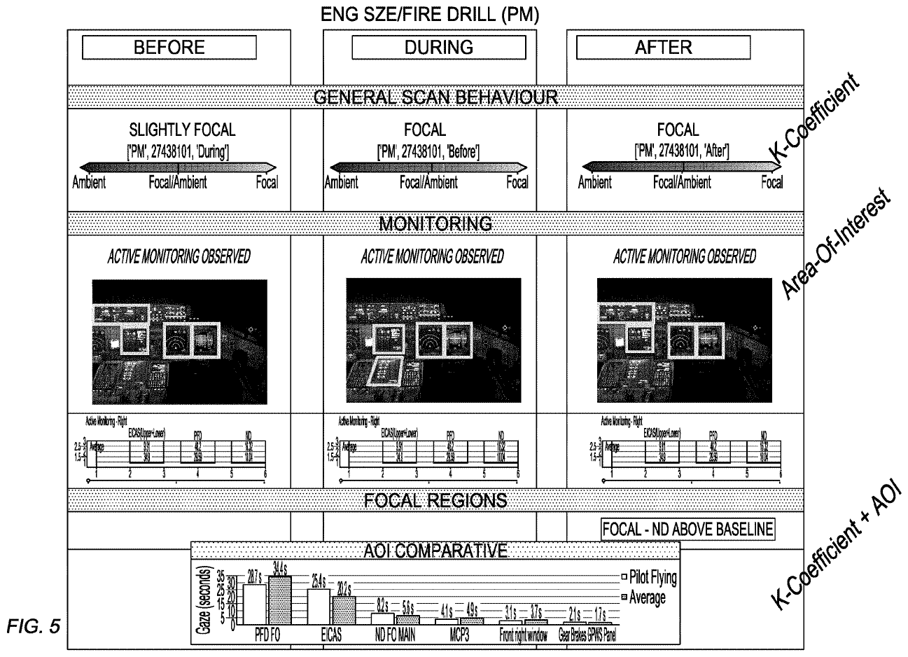

FIG. 5 is an example of a user interface that may be displayed to the student and/or instructor after a simulation. In this particular example, the flight simulation subjects the student to a simulated engine fire and engine seizure. During the simulation, the student reacts to the event (in this case the engine fire and seizure) by performing various tasks such as shutting down the engine, activating a fire bottle (fire suppression), as well as communicating with the other members of the crew. The biometry-based performance assessment uses eye tracking data and optionally voice/speech, facial expressions and other non-intrusive biometry to assess the student's reaction to the emergency event. Once the simulation is over, the student and instructor can debrief using the user interface presented in FIG. 5. The user interface of FIG. 5 reveals various biometry-based performance aspects before, during and after the event. The UI of FIG. 5 shows general scan behavior before, during and after the event. In this particular example, the UI displays a K-coefficient analysis that is indicative of how focal or ambient the student's gaze was before, during and after the event. The UI of FIG. 5 also displays the student's instrument monitoring performance by highlighting the areas of interest upon which the student was fixated before, during and after the event. The UI of FIG. 5 may also present a comparative analysis showing how the focal regions of the student compare with baselines, group averages or medians, prescribed standards, or to expected or ideal instrument scanning performance.

For the purposes of interpreting this specification, when referring to elements of various embodiments of the present invention, the articles “a”, “an”, “the” and “said” are intended to mean that there are one or more of the elements. The terms “comprising”, “including”, “having”, “entailing” and “involving”, and verb tense variants thereof, are intended to be inclusive and open-ended by which it is meant that there may be additional elements other than the listed elements.

This invention has been described in terms of specific implementations and configurations which are intended to be exemplary only. Persons of ordinary skill in the art will appreciate that many obvious variations, refinements and modifications may be made without departing from the inventive concepts presented in this application. The scope of the exclusive right sought by the Applicant(s) is therefore intended to be limited solely by the appended claims.

1. A computerized system for assessing performance based on biometry, the system comprising:
one or more processors executing an artificial intelligence module for correlating, during a model training phase, low-resolution biometric sensor training data obtained from a low-resolution biometric sensor with a cognitive load index generated from high-resolution biometric sensor data to train a cognitive load model;
wherein the one or more processors are configured to assess performance during an operational phase by:
obtaining low-resolution biometric sensor operational data from the low-resolution biometric sensor during the operational phase; and
determining a cognitive load during the operational phase based on the low-resolution biometric sensor operational data and the cognitive load model.

2. The system of claim 1 comprising a low-resolution biometric sensor selected from a group consisting of an eye tracker, facial muscular movement sensor, low-resolution electroencephalogram (EEG), low-resolution thermal brain imager and low-resolution galvanometer of endodermal activity (EDA) sensor.

3. The system of claim 1 comprising a high-resolution biometric sensor selected from a group consisting of a high-resolution EEG, a Functional Near-Infrared Spectroscopy (FNIRS) sensor, a high-resolution thermal brain imager, a high-resolution galvanometer of endodermal activity (EDA) and an electrocardiogram (ECG).

4. The system of claim 1 wherein the low-resolution biometric sensor is an eye tracker.

5. The system of claim 4 wherein the eye tracker tracks a gaze to provide gaze data and wherein the eye tracker further comprises a pupillometer to provide pupil dilation data.

6. The system of claim 5 wherein the gaze data includes gaze pattern data, fixation data and saccade data.

7. The system of claim 1 wherein the one or more processors execute an emotion inference module to infer an emotional state based on a facial expression.

8. The system of claim 1 wherein the one or more processors execute an emotion inference module to infer an emotional state based on one or both of voice quality and speech content.

9. The system of claim 1 wherein the one or more processors execute an emotion inference module to infer an emotional state based on facial muscular movement data.

10. The system of claim 1 wherein the one or more processors generate a learner profile that correlates biometrically determined cognition levels with ability to learn to perform different types of tasks.

11. The system of claim 1 wherein the one or more processors executed an augmented cognition module to automate a task otherwise performed by a user in response to detecting a lowered cognitive level of the user.

12 A computer-implemented method of assessing performance based on biometry, the method comprising:
correlating during a model training phase, by an artificial intelligence module, low-resolution biometric sensor training data obtained from a low-resolution biometric sensor with a cognitive load index generated from high-resolution biometric sensor data to train a cognitive load model; and
assessing performance during an operational phase by:
obtaining low-resolution biometric sensor operational data from the low-resolution biometric sensor during the operational phase; and
determining a cognitive load during the operational phase based on the low-resolution biometric sensor operational data and the cognitive load model.

13. The method of claim 12 comprising obtaining the low-resolution biometric sensor training data using a low-resolution biometric sensor selected from a group consisting of an eye tracker, facial muscular movement sensor, low-resolution electroencephalogram (EEG), low-resolution thermal brain imager and low-resolution galvanometer of endodermal activity (EDA) sensor.

14. The method of claim 12 comprising obtaining the high-resolution biometric sensor data using a high-resolution biometric sensor selected from a group consisting of a high-resolution EEG, a Functional Near-Infrared Spectroscopy (FNIRS) sensor, a high-resolution thermal brain imager, a high-resolution galvanometer of endodermal activity (EDA) and an electrocardiogram (ECG).

15. The method of claim 12 wherein the low-resolution biometric sensor is an eye tracker.

16. The method of claim 15 wherein the eye tracker generates eye tracking data comprises obtaining gaze data from the eye tracker and obtaining pupil dilation data from a pupillometer.

17. The method of claim 16 wherein the gaze data includes gaze pattern data, fixation data and saccade data.

18. The method of claim 12 comprising using an emotion inference module to infer an emotional state based on a facial expression.

19. The method of claim 12 comprising using an emotion inference module to infer an emotional state based on one or both of voice quality and speech content.

20. The method of claim 12 wherein the one or more processors execute an emotion inference module to infer an emotional state based on facial muscular movement data.

1. The method of claim 12 comprising generating a learner profile that correlates biometrically determined cognition levels with ability to learn to perform different types of tasks. Gowling WLG (Canada) LLP Page 4

22. The method of claim 12 comprising an augmented cognition module to automate a task otherwise performed by a user in response to detecting a lowered cognitive level of the user.

23. A non-transitory computer-readable medium having instructions in code which are stored on the computer-readable medium and which, when executed by one or more processors of one or more computers, cause the one or more processors to assess performance based on biometry by:
correlating during a model training phase, by an artificial intelligence module, low-resolution biometric sensor training data obtained from a low-resolution biometric sensor with a cognitive load index generated from high-resolution biometric sensor data to train a cognitive load model; and
assessing performance during an operational phase by:
obtaining low-resolution biometric sensor operational data from the low-resolution biometric sensor during the operational phase; and
determining a cognitive load during the operational phase based on the low-resolution biometric sensor operational data and the cognitive load model.

24. The non-transitory computer-readable medium of claim 23 wherein the low-resolution biometric sensor training data is obtained using a low-resolution biometric sensor selected from a group consisting of an eye tracker, facial muscular movement sensor, low-resolution electroencephalogram (EEG), low-resolution thermal brain imager and low-resolution galvanometer of endodermal activity (EDA) sensor.

25. The non-transitory computer-readable medium of claim 23 wherein the high-resolution biometric sensor data is obtained using a high-resolution biometric sensor selected from a group consisting of a high-resolution EEG, a Functional Near-Infrared Spectroscopy (FNIRS) sensor, a high-resolution thermal brain imager, a high-resolution galvanometer of endodermal activity (EDA) and an electrocardiogram (ECG).

26. The non-transitory computer-readable medium of claim 23 wherein the low-resolution biometric sensor training data is obtained from an eye tracker that obtains gaze data and pupil dilation data.

27. The non-transitory computer-readable medium of claim 26 wherein the gaze data includes gaze pattern data, fixation data and saccade data.

28. The non-transitory computer-readable medium of any claim 23 comprising code for an emotion inference module to infer an emotional state based on a facial expression.

29. The non-transitory computer-readable medium of any claim 23 comprising code for an emotion inference module to infer an emotional state based on one or both of voice quality and speech content.

30. The non-transitory computer-readable medium of any claim 23 wherein the one or more processors execute an emotion inference module to infer an emotional state based on facial muscular movement data.

31. The non-transitory computer-readable medium of any claim 23 comprising code for generating a learner profile that correlates biometrically determined cognition levels with ability to learn to perform different types of tasks.

32. The non-transitory computer-readable medium of any claim 23 comprising code for an augmented cognition module to automate a task otherwise performed by a user in response to detecting a lowered cognitive level of the user.
